(19) United States
(12) Patent Application Publication
(10) Pub. No.: US 2010/0247642 A1
(43) Pub. Date: Sep. 30, 2010

(54) STABLE PHARMACEUTICAL FORMULATION FOR A DPP-IV INHIBITOR

(75) Inventors: Zhen-Ping Wu, La Jolla, CA (US); Richard Alexander Moore, JR., San Diego, CA (US)

Correspondence Address: SCHWEGMAN, LUNDBERG & WOESSNER, P.A., P.O. BOX 2938, MINNEAPOLIS, MN, 55402, US

(73) Assignee: Phenomix Corporation, San Diego, CA (US)

(21) Appl. No.: 12/600,941

(22) PCT Filed: May 21, 2008
(86) PCT No.: PCT/US08/64363

§ 371 Date: May 17, 2010

Related U.S. Application Data
(60) Provisional application No. 60/939,292, filed on May 21, 2007.

(51) Int. Cl. A61K 9/32; A61K 31/69; A61P 3/08

(52) U.S. Cl. 424/465; 514/64

(57) ABSTRACT

A dosage form is provided for an anti-diabetic DPP-IV inhibitor of formula (I) as its tartarate salt, wherein the purity of the active pharmaceutical ingredient is maintained over a prolonged storage period under conditions similar to those likely encountered in home storage of the medication by a diabetic patient. A formulation free of calcium salts such as calcium phosphate, but including microcrystalline cellulose, copovidone, crospovidone, colloidal silicon dioxide, and magnesium stearate, when compacted into a tablet with the active pharmaceutical ingredient, was shown to be stable for at least six months at 40° C. and 75% relative humidity. Methods for preparation of the dosage form are also provided.

### CROSS-REFERENCE TO RELATED APPLICATIONS

This application claims the priority of U.S. Ser. No. 60/939,292, filed May 21, 2007, which is incorporated herein by reference in its entirety.

### FIELD OF THE INVENTION

The field of the invention is a tablet dosage form for an inhibitor of dipeptidyl peptidase IV that has a high degree of stability, including under warm, humid storage conditions.

### BACKGROUND

The enzyme dipeptidyl peptidase IV (DPP-IV) is a member of the dipeptidyl peptidase family, which cleaves N-terminal dipeptide residues from proteins, particularly where the dipeptide includes an N-terminal penultimate proline or alanine residue. DPP-IV is believed to be involved in glucose control, as its peptidolytic action inactivates the insulotropic peptides glucagon-like peptide I (GLP-1) and gastric inhibitory protein (GIP). Inhibition of DPP-IV, such as with synthetic inhibitors in vivo, can serve to increase plasma concentrations of GLP-1 and GIP, and thus improve glycemic control in the body. Such synthetic inhibitors would therefore be useful in the treatment of Diabetes Mellitus and related conditions.

However, there exist other members of this DPP enzyme family including DPP-XII, DPP-XIII, DPP-IX, and FAP (fibroblast activation protein), which have similar substrate specificities to DPP-IV. Inhibition of certain of these enzymes, for example DPP-XIII, is known to cause toxic effects in mammals. Therefore, to be medicinally useful, inhibitors of DPP-IV must also exhibit selectivity for DPP-IV relative to at least some other members of the DPP enzyme family.

Certain such selective DPP-IV inhibitors have been developed, as is disclosed in the published PCT patent application, publication number WO2005/047297 and in U.S. Application Publication Nos. 2006/0258621, 2006/0264400, and 2006/0264401. Inhibition of DPP-IV by compounds of the structure of formula (I):

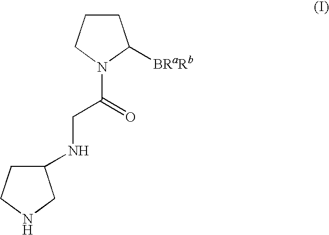

wherein R^{a} and R^{b} are OH providing a boronic acid, or its salt or a protected form, is disclosed therein. The compound is referred to as a pyrrolidin-3-yl-glycyl-boro-proline, or more generally, a pyrrolidin-3-ylglycylaminoalkylboronate. U.S. Pub. No. 2006/0264400, published Nov. 23, 2006, specifically claims a compound of this structure and its use for selectively inhibiting DPP-IV, such as in a mammal with a malcondition that can be regulated or normalized by inhibition of DPP-IV, such as diabetes.

In order to obtain the benefits of administration of a selective DPP-IV inhibitor, particularly by oral ingestion, a patient must ingest the inhibitor compound in a form adapted to facilitate absorption of the active pharmaceutical ingredient into the blood stream where it can be transported to the site of action within the body. The dosage form, which in some applications will likely be adapted for home use on a daily or other regular basis by diabetic patients, must also provide for stability of the bioactive compound under the storage conditions typically encountered in patients' homes, for example, in a home medicine cabinet where exposure to warmth and humidity is expected. Therefore, there is a need for a dosage form for a selective DPP-IV inhibitor that provides for thorough and rapid dissolution of the dosage form within the body, facilitating uptake of the active pharmaceutical ingredient by the patient, while also providing for stability of the dosage form under likely storage conditions, as in the medicine cabinets of patients prescribed the drug.

### SUMMARY OF THE INVENTION

The invention is directed to a dosage form for a DPP-IV inhibitor that provides for a surprisingly high degree of storage stability, particularly under warm or humid conditions. An embodiment of the present invention is directed to a tablet dosage form for the active pharmaceutical ingredient having formula (I):

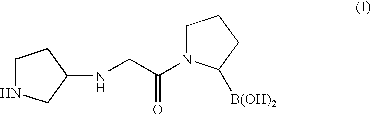

as its tartarate salt. This is understood to include any solvates, hydrates, tautomers or stereoisomers thereof. Any stereoisomeric form of a compound of formula (I), and optical isomeric mixtures, are included. The dosage form comprises a tartarate salt of the compound of formula (I); a diluent comprising a microcrystalline cellulose; a binder comprising copovidone; a disintegrant comprising crospovidone; a lubricant comprising magnesium stearate; and a glidant comprising colloidal silicon dioxide. The tartarate salt of the compound of formula (I) can be a monotartarate, an L-tartarate, or both. The dosage form is free of a calcium salt. More specifically, the dosage form is free of calcium phosphate.

The compound of formula (I) is an inhibitor of the enzyme dipeptidyl peptidase IV (DPP-IV). More particularly, a specific stereoisomer of this compound, a compound of formula (II)

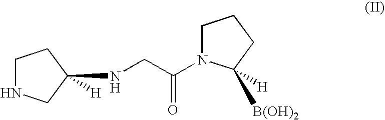

is an inhibitor of DPP-IV, and in the form of a tartarate salt is likewise an inhibitor of DPP-IV. An embodiment of the present invention is directed to the dosage form recited above including the specific stereoisomer of formula (II) as a tartarate salt.

An embodiment of the present invention, directed to a method of preparation of the inventive dosage form, involves milling the compound of formula (I) tartarate salt to provide a milled compound; then, blending the milled compound with a diluent including microcrystalline cellulose to provide a blended milled compound; then in a fluidized bed granulator, granulating the blended milled compound with a solution of the binder including copovidone in water to provide granules; then drying the granules; then milling and screening the granules to provide dried, milled granules; then blending the dried, milled granules with the dispersant including crospovidone, the glidant including colloidal silicon dioxide, and the lubricant including magnesium stearate, to provide a lubricated blend; then compressing the lubricated blend in a tablet press to provide the inventive dosage form. The dosage form is free of a calcium salt. More specifically the dosage form is free of calcium phosphate.

Another embodiment of a method of preparation of the inventive dosage form involves dry mixing the compound of formula (I) tartarate, the diluent including microcrystalline cellulose, and the binder including copovidone, in a high shear granulator to provide a dry mix; then adding water to the dry mix to provide granules; then drying and milling the granules; then adding the dispersant including crospovidone, the glidant including colloidal silicon dioxide and the lubricant including magnesium stearate; then mixing all these together to provide a lubricated blend; then compressing the lubricated blend in a tablet press to provide the inventive dosage form. Again the dosage form is free of a calcium salt; more specifically, the dosage form is free of calcium phosphate.

Yet another embodiment of a method of preparation of the inventive dosage form involves dry granulating a combination of the compound of formula (I) and diluent including microcrystalline cellulose using a technique such as roller compacting. The resulting dry granules are milled or ground into a powder and the powder is combined with dispersant, glidant and lubricant as described above. The resulting lubricated blend is then compressed into tablets to provide the inventive dosage form.

The inventive dosage form can include from about 50 to about 500 mg of the compound of formula (I) tartarate on a free base basis. Specifically, the inventive dosage form can include about 50 mg, about 100 mg, about 200 mg, or about 400 mg of the inventive compound on a free base basis.

### DETAILED DESCRIPTION OF THE INVENTION

### Definitions

A “dosage form” as used herein refers to a physical and chemical composition of an active pharmaceutical ingredient (API) that is adapted for administration to a patient in need thereof. The inventive dosage form is a tablet. By a tablet is meant a relatively hard, compact object, suitable for oral ingestion, prepared by compression of a powder including an active pharmaceutical ingredient and, usually, excipients. An “excipient” is an ingredient of the dosage form that is not medicinally active, but serves to dilute the API, assist in dispersion of the tablet in the patient's stomach, bind the tablet together, and serve other functions like stabilizing the API against decomposition.

The inventive tablet can be coated or uncoated. By “coated” is meant that the tablet is covered with a layer, usually a continuous layer, of a substance such as a polymer including but not limited to polyvinyl pyrrolidone (PVA), hydroxypropyl methyl cellulose (HPMC) and/or hypromellose that can serve to preserve tablet integrity, reduce dusting, and repel moisture. Such coatings are typically termed moisture-protective coatings. An uncoated tablet lacks the covering layer, thus exposing the core to environmental conditions.

The processes of preparing the inventive dosage form including milling, screening, drying, blending, granulation, etc. are carried out as is well-known in the art, as described in Remington: The Science and Practice of Pharmacy, 21^{St} edition, Lippincott, Williams & Wilkins, (2005), which is incorporated herein by reference. Terms as are used in the compounding arts, such as granulation and fluidized bed granulation (also known as fluid bed granulation), are described in detail therein. As used herein, “high shear” granulation refers to a dry granulation process carried out with a relatively high degree of shear forces being applied to the solids during the granulation process, for example during mixing prior to addition of the water in the formation of granules from a mixed powder including the active pharmaceutical ingredient and excipients. High shear forces aid in dispersion of the active pharmaceutical ingredient, usually as a powder of relatively fine texture, with the excipients.

An “active pharmaceutical ingredient,” or API, is a molecular entity adapted for treatment of a malcondition in a patient in need thereof. The present active pharmaceutical ingredient in an inhibitor of the enzyme DPP-IV, which can be useful in the treatment of diabetes and other conditions involving the need for improvement in glycemic control. The API of the present invention is an aminoboronic acid, which is present in the inventive dosage form as its tartarate salt. By a “tartarate” is meant herein a salt of tartaric acid. The tartaric acid can be of any stereochemical configuration, or any mixture thereof. For example, a tartarate salt of the invention can be a salt of D-tartaric acid, L-tartaric acid, DL-tartaric acid, meso-tartaric acid, or any combination thereof.

A “diluent” is a pharmacologically inert substance that is nevertheless suitable for human consumption, that serves as an excipient in the inventive dosage form. A diluent serves to dilute the API in the inventive dosage form, such that tablets of a typical size can be prepared incorporating a wide range of actual doses of the API. A diluent can comprises a microcrystalline cellulose, for example, Avicel. Lactose and isomalt are other common diluents. Avicel, a form of microcrystalline cellulose, is a commercially available product that is formed of acid-treated cellulose, which treatment tends to dissolve more amorphous regions of the cellulose and to leave more crystalline regions of the cellulose. Microcrystalline cellulose is a diluent in the inventive dosage form.

Other diluents well-known to those skilled in the art include monobasic calcium phosphate, dibasic calcium phosphate and tribasic calcium phosphate. Almost completely water-insoluble, calcium phosphates are particularly well-known pharmacologically inert diluents or fillers that are compatible with a wide range of APIs. By the term “calcium phosphate” is meant herein calcium phosphate in any of its forms, including monobasic calcium phosphate (Ca(H₂PO₄)₂)), dibasic calcium phosphate (CaHPO₄) and tricalcium phosphate (Ca₂(PO₄)₃), including any orthophosphates, pyrophosphates or superphosphates, or other polymeric phosphates wherein the counterion includes calcium. By a “calcium salt” is meant any ionic compound including calcium, specifically including the above-listed calcium phosphates, and calcium sulfate.

A “binder” is a pharmacologically inert substance, suitable for human consumption, that serves to hold the constituents of a tablet together after compression forming of the tablet has occurred. Copovidone is a binder in the inventive dosage form. By “copovidone,” also known as “copolyvidone,” is meant a copolymer of vinyl pyrrolidone and vinyl alcohol, as is well-known in the art. The copolymer can be a graft copolymer. When used as a binder, the copovidone provides good adhesion, elasticity, and hardness, and may assist in repelling moisture from the tablets, once formed.

A “disintegrant” is a substance that assists in dissolution of the dosage form after oral ingestion. It is believed to assist in hydration and to avoid the formation of gels in the stomach of the patient as the tablet dissolves, thus assisting in the release of the API into the gastric juices so that it can be absorbed into the bloodstream. The disintegrant of the inventive dosage form includes crospovidone, a cross-linked polyvinylpyrrolidone.

A “glidant” is a substance that assists in maintaining favorable powder flow properties of the powder materials that are compressed to form the inventive tablet. The glidant of the present invention includes colloidal silicon dioxide, which is a fumed silica with a particle size of about 15 nm.

A “lubricant” is a substance that is useful in the tablet compression process, serving to lubricate metal parts of the tablet die. The lubricant of the present invention includes magnesium stearate.

A “free base” is the molecular form of an amine wherein the amine is not in salt form. When it is stated that an inventive dosage form contains some quantity of the compound of formula (I) tartarate “on a free base basis,” what is meant is that the quantity of the tartarate salt form of the API that is included is equivalent to the stated quantity of the API in its free base form; i.e., that actual quantity of API tartarate in the dosage form is normalized for the difference in molecular weight between the free base and the tartarate salt of the free base of the compound of formula (I). Thus, for a monotartarate, non-hydrated form, the actual weight of the tartarate salt will be about 162% of the weight of the API on a free base basis, the ratio of the sum of the molecular weights of the compound of formula (I) and tartaric acid to the molecular weight of the compound of formula (I), i.e., about 390/240.

The stability of an API in a dosage form can be expressed by providing data concerning the percent decomposition of the API that occurs over a certain time period, when the dosage form is stored at a stated temperature and relative humidity (RH). This value can be expressed as the percent of remaining API, or as the ratio of the purity of the API at the given time point over the purity of the API at the beginning of the time period ending in that time point. By relative humidity is meant the percent of water saturation of the air at the stated temperature.

### DETAILED DESCRIPTION

The present invention is directed to a dosage form for an API, wherein the API is a tartarate salt of a compound of formula (I) as defined herein. The compound of formula (I) is an aminoboronic acid analog of a peptide that inhibits the bioactivity of the enzyme DPP-IV. The compound of formula (I) is a selective inhibitor of DPP-IV that can be used for treatment of a malcondition involving glycemic control, such as takes place in diabetes. Other malconditions involving glycemic control include hyperglycemia and hypoglycemia. The inventive dosage form has been unexpectedly found to provide for greater API stability than would a dosage form for the API that a person of ordinary skill in the art would likely select.

The compound of formula (I) is disclosed and claimed in U.S. Pub. No. 2006/0264400 by the inventors herein. The tartarate salt of a compound of formula (I) and formulations thereof are disclosed and claimed in U.S. Ser. No. 60/841,097 by the inventors herein. The present invention discloses and claims a dosage form adapted for administration of the tartarate salt of the compound of formula (I), wherein the inventors have surprisingly found that the API is more stable on prolonged storage under typical storage conditions than is the same API when formulated in a standard manner. This was unexpectedly found to be the case even when the API is in an uncoated tablet dosage form, provided that the excipients include the ingredients claimed herein and exclude calcium salts. Common calcium salts used as excipients include calcium phosphates and calcium sulfate.

A comparison of the stability of the API of the current invention was made, using excipients well-known in the art. Table 1 shows the results of stability studies on a binary mixture of the API herein plus dibasic calcium phosphate. Compound purity was determined by HPLC. The mixture of the API and the calcium phosphate was allowed to stand under the specified conditions for the stated times. Results are given as percent purity of the API at the given time point.

TABLE 1
Excipient Compatibility Results for API and Dibasic Calcium
Phosphate

| | Time | | | | |
|---|---|---|---|---|---|
| | | 2 weeks/ | 2 weeks/ | 4 weeks/ | 8 weeks/ |
| conditions | Initial | loose cap | tight cap | tight cap | tight cap |
| % API | 89.2 | 77.0 | 77.2 | 71.7 | 54.7 |

Initially, at time 0, the starting API purity was found to be about 90%. Within two weeks, regardless of the amount of exposure of the mixture to atmospheric conditions, purity had dropped by over 10%, and by 8 weeks, even in a sealed vial, the purity was barely above 50%.

In contrast, Table 2 shows a binary mixture stability study of another well-known diluent, microcrystalline cellulose. Again, the mixture of the API and the microcrystalline cellulose was allowed to stand under the specified conditions for the stated times. Results are given as percent purity of the API at the given time point.

TABLE 2
Excipient Compatibility Results for API and Microcrystalline
Cellulose

Time/conditions

| | | 2 weeks/ | 4 weeks/ | 8 weeks/ |
|---|---|---|---|---|
| | Initial | loose cap | tight cap | tight cap |
| % API | 90.5 | 82.1 | 90.2 | 91.7 |

Again, the starting purity of the API was about 90%, but in this case, even at 8 weeks storage, the purity was substantially unchanged.

It is generally understood in the art that microcrystalline cellulose and dibasic calcium phosphate are about equally suitable for use as diluents or fillers in pharmaceutical compositions. Both are generally regarded as inert substances that are suitable for formation of tablets containing API substances by compression in tablet presses. For example, in Remington it is stated (page 902, 21^{st} Edition), that “Direct-compression vehicles or carriers must have good flow and compressible characteristics . . . . The vehicles include processed forms of most of the common diluents including dicalcium phosphate dihydrate, tricalcium phosphate, calcium sulfate, anhydrous lactose, spray-dried lactose, pregelatinized starch, compressible sugar, mannitol, and microcrystalline cellulose.” Thus, if a person of ordinary skill were consulting a formulation encyclopedia such as Remington, the person would be led to the conclusion that calcium phosphate and microcrystalline cellulose, along with lactose or mannitol, would be equally suitable as carriers for their API.

Furthermore, as the inhibitor of DPP-IV is adapted for treatment of malconditions involving glycemic control, such as diabetes, a person of ordinary skill would be expected to select a diluent that was other than a sugar, sugar alcohol, or a substance like a sugar that can act as a substrate either for human sugar-transporting or metabolizing enzymes or for gastro-intestinal bacterial populations. Diabetic patients typically need to maintain strict control of carbohydrates in their diet, which would lead a person of ordinary skill to select compounds like dicalcium phosphate dihydrate, tricalcium phosphate, calcium sulfate or microcrystalline cellulose, rather than any of the usual metabolizable carbohydrate excipients like lactose or mannitol.

However, the inventors herein have surprisingly discovered that calcium phosphate has a markedly detrimental effect on the storage stability of the API in the present invention. As shown above, the presence of calcium phosphate causes massive decomposition of the compound of formula (I) tartarate, including over periods of time and under conditions similar to those that would be expected to be encountered on storage of self-administered anti-diabetes drugs in patients' medicine cabinets. Particularly as the inventive drugs are expected to be useful for the oral treatment of diabetes, wherein diabetic patients will keep substantial reserves of the drug on hand (as withdrawal could be life-threatening) and would also be expected to self-administer the drug, for example on a daily basis (so it would be stored in home environments), this discovery of the API's instability in the presence of a common excipient is significant.

Table 3 shows the results of a long-term stability study of tablets including the inventive API using a series of excipients suitable for the purpose as discovered herein and excluding calcium salts. A dosage form lacking calcium phosphate, but including microcrystalline cellulose, was prepared by forming tablets including these ingredients as well as others known to be useful as excipients. The inventive dosage form thus includes microcrystalline cellulose, copovidone, crospovidone, colloidal silicon dioxide, and magnesium stearate, but excludes calcium phosphate. Additionally, the inventive dosage form may include a tablet coating such as Eudragit® (sold by Degussa) or Opadry® (sold by Color Con).

The tablets used in this study, each containing 400 mg of the API on a free base basis (FBB), were prepared according to a method of the invention (fluidized bed granulation), and were a composition of the invention. The tablets were stored and exposed to the atmosphere under the given conditions for the periods of time indicated in the Table. Each tablet was then extracted and analyzed by HPLC to determine how much, if any, decomposition of the API had taken place.

TABLE 3
Six Month 400 mg Tablet Prototype Stability Study--% API
Remaining

| Conditions | Uncoated | Coated |
|---|---|---|
| 25° C./60% RH | 105.3 | — |
| 3 months | | |
| 25° C./60% RH | 106 | 107.8 |
| 6 months | | |
| 40° C./75% RH | 98.2 | — |
| 2 weeks | | |
| 40° C./75% RH | 95.9 | — |
| 4 weeks | | |
| 40° C./75% RH | 104.8 | — |
| 3 months | | |
| 40° C./75% RH | 105.3 | 105.5 |
| 6 months | | |

The purity of the starting material was about 100%. All the values in table 3 are statistically indistinguishable from 100% by the analytical methods used. As can be seen, even after six months storage at 40° C. and 75% RH, no significant decomposition of the API was observed, as the percent API remaining of all tested samples were statistically indistinguishable, thus confirming the suitability of the claimed dosage form for prolonged storage of the compound of formula (I) tartarate salt.

The dosage form can be prepared to contain substantially any quantity less than about 500 mg of the API on a free base basis. For example, the dosage form can contain about 50 mg, 100 mg, about 200 mg, or about 400 mg of the API on a free base basis. Examples of 200 mg and 400 mg dosage forms are provided below in the Examples.

It is surprising that the manufacturing process of the invention that utilizes direct compression and avoids wet granulation will form tablets of the invention containing high amounts of the formula I tartrate. The physical properties of high-load APIs are not conducive to simple and manufacturable formulations. Acetominophen, for example, possesses very poor compression characteristics, requiring a precursor step in manufacturing (such as granulation) or the use of substantial volumes of excipients to render the material manufacturable in tablet form. While ibuprofen and guafenesin are more readily compressible, they exhibit the feature of exceptionally low-melting points which feature is manifested by the compressed formulation sticking and picking to the punch faces of a tablet press. Accordingly, Mr. Hite, a formulations expert, reports in “Drug Delivery Technology” http://www.drugdeliverytech-online.com/drugdelivery/200705/?pg=32 that drug loading exceeding 30% is well known to require precursor processes such as wet granulation to impart compressibility characteristics. Thus, according to the invention the ability to produce tablets of high API content by a dry, direct compression technique is surprising, especially when that content reaches or exceeds 70% by weight.

Certain non-limiting examples are provided below to illustrate embodiments of the inventive methods. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

### EXAMPLES

### Example 1

### Manufacture of 200 mg Tablets Using a Fluid Bed Granulation Technique

For the preparation of a batch of tablets of the inventive dosage form, containing 200 mg each of the API on a free base basis, the following procedure was used:

A sample of the compound of formula (I) tartarate salt, (5.35 kg) was placed in a Fitzmill L1A with screen 0033 in place. The mill was operated at 3015 rpm, and 5.35 kg of milled compound (loss=5.0 gm) that passed the screen was collected in a polyethylene bag in the presence of a desiccant. Then microcrystalline cellulose PH112 (5.97 kg, previously screened through 16 mesh screen) was placed in the Fitzmill with the 0033 screen and processed at 3010 rpm through the screen. Then, a solution of copovidone (625 mg, Kolva 64 fine) was dissolved in 1.90 kg purified water in a caframo vixed at 1200 rpm for 30 min. Into a 32 qt. V-shell blender, preheated to 55-65° C., at 25 rpm was added 5.09 kg of the milled compound of formula (I) tartarate, 5.97 kg of the microcrystalline cellulose, and the mixture blended for 15 min. Then, an Aeromatic S-2 Fluid Bed granulator fitted with a 1.2 mm nozzle, a peristaltic pump, and a 200 mesh bottom screen was set up, and the solution spray system charged with the copovidone solution. The inlet air temperature was set at 60±7° C. and the atomizing pressure at 2.0 bar. Air flow was 74-143 cfm. The mixture of the milled compound of formula (I) tartarate and the microcrystalline cellulose as charged into the bowl and blended at very low fluidization air velocity for 3 min, then spraying of the solution of copovidone in water at a spray rate of 50±10 gm/min was commenced. After the solution had been completely added (1 hr) followed by an addition 0.30 kg water. Then, after spraying was complete, the granulation was dried in the Aeromatic S2 fluid bed dryer at an inlet temperature of 60±7° C. to a target moisture content of 4.5%. Drying was stopped when the outlet air temperature reached 42±3° C., to yield 11.7221 kg of dried granules. The dried granules were then passed through a Quadro Comil 1975 fitted with a 045R or 055R round screen and round impeller, set at 1450 rpm, to provide 11.7005 kg of dried milled granules, which were kept in the presence of a desiccant. A repeat of this entire above procedure with a second batch yielded 11.5836 kg of the dried milled granules. The two batches were combined in a 5 cu-ft V-blender and blended for 10 min. Then, crospovidone (1.225 kg, XL10) and colloidal silicon dioxide (245.0 gm) were added and the mixture blended 10 min, followed by magnesium stearate (122.5 gm) which was blended in an additional 3 min to provide the inventive lubricated blend. For the theoretical batch size of 25.0 kg, 24.8 (99.2%) was recovered. A Manesty Betapress Piccola rotary tablet press equipped with 0.7480″×0.370″ upper and lower capsule-shaped punches was set up with a nominal compression force of 13.1 kN. The press was set up to operate at 5 stations at a rate of 175 tpm. A total intact tablet weight of 23.9327 kg was obtained of tablets each containing a nominal 200 mg each of the API on a free base basis.

### Example 2

### Manufacture of 400 mg Tablets Using a Fluid Bed Granulation Technique

For the preparation of a batch of tablets of the inventive dosage form, containing 200 mg each of the API on a free base basis, the following procedure was used:

A sample of the compound of formula (I) tartarate salt, (10.71) was placed in a Fitzmill L1 A with screen 0033 in place. The mill was operated at 3005 rpm, and 9.626 kg of milled compound that passed the screen was collected in a polyethylene bag in the presence of a desiccant. Then microcrystalline cellulose PH112 (2.084 kg, previously screened through 16 mesh screen) was placed in the Fitzmill with the 0033 screen and processed at 3006 rpm through the screen. Then, a solution of copovidone (625 mg, Kolva 64 fine) was dissolved in 1.90 kg purified water in a caframo vixed at 1200 rpm for 30 min. Into a 32 qt. V-shell blender, preheated to 55-65° C., at 25 rpm was added 5.09 kg of the milled compound of formula (I) tartarate, 5.97 kg of the microcrystalline cellulose, and the mixture blended for 15 min. Then, an Aeromatic S-2 Fluid Bed granulator fitted with a 1.2 mm nozzle, a peristaltic pump, and a 200 mesh bottom screen was set up, and the solution spray system charged with the copovidone solution. The inlet air temperature was set at 60±7° C. and the atomizing pressure at 2.0 bar. Air flow was 74-143 cfm. The mixture of the milled compound of formula (I) tartarate and the microcrystalline cellulose as charged into the bowl and blended at very low fluidization air velocity for 3 min, then spraying of the solution of copovidone in water at a spray rate of 25±10 gm/min was commenced. After the solution had been completely added (1 hr) followed by an addition 0.30 kg water. Then, after spraying was complete, the granulation was dried in the Aeromatic S2 fluid bed dryer at an inlet temperature of 60±7° C. to a target moisture content of less than 8%. Drying was stopped when the outlet air temperature reached 42±3° C., to yield 12.249 kg of dried granules. The dried granules were then passed through a Quadro Comil 197S fitted with a 045R or 055R round screen and round impeller, set at 1400 rpm, to provide 12.221 kg of dried milled granules, which were kept in the presence of a desiccant. A repeat of this entire above procedure with a second batch yielded 12.194 kg of the dried milled granules. The two batches were combined in a 5 cu-ft V-blender and blended for 10 min. Then, crospovidone (1.24 kg, XL10) and colloidal silicon dioxide (247.5 gm) were added and the mixture blended 10 min, followed by magnesium stearate (123.8 gm) which was blended in an additional 3 min to provide the inventive lubricated blend. For the theoretical batch size of 26.25 kg, 25.91 (98.7%) was recovered. A Manesty Betapress Piccola rotary tablet press equipped with 0.7480″×0.370″ upper and lower capsule-shaped punches was set up with a nominal compression force of 23 kN. The press was set up to operate at 5 stations at a rate of 200 tpm. A total intact tablet weight of 24.772 kg was obtained of tablets each containing a nominal 400 mg each of the API on a free base basis.

### Example 3

### Manufacture of Tablets Using a Dry Granulation and Direct Compression Technique

A process for making tablets of the invention using the current formulation described in Example 5 but without using high-shear wet granulation, fluid bed granulation or direct compress of dry powder bled can be accomplished as follows:

The active ingredient along with portions (or all) of the following ingredients: microcrystalline cellulose, copovidone, crospovidone, colloidal silicon dioxide and magnesium stearate are mixed together in stepwise fashion to produce a uniform blend using a series of blender or screening mill steps. The resulting blend is then compacted into ribbons or slugs or pellets using either roller compaction or a tablet press. The resulting compacts are then milled into granules using a screening mill or hammer mill and blended together with remaining portions (or all) of the following ingredients: microcrystalline cellulose, copovidone, crospovidone, colloidal silicon dioxide and magnesium stearate. The resulting blend is then compacted on a rotary tablet press to produce tablets which can then be film coated.

### Example 4

Manufacture of Tablets Using A Dry Powder Direct Compression Technique

| | Unit | | |
|---|---|---|---|
| Step | Operation | Manufacturing Process | Components |
| I. | DISPENSE | 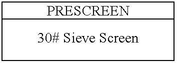 | A. Microcrystalline Cellulose B. Copovidone C. Crospovidone D. Magnesium Stearate E. Colloidal Silicon Dioxide (16#) |
| II. | PREBLEND | 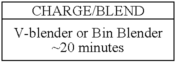 | Add: 1. Formula I monotartarate (assay adjusted 2. Microrystalline Cellulose 3. Copovidone |
| III. | BLEND | 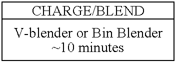 | Add: 4. Crospovidone 5. Colloidal Silicon Dioxide (Screen 16#) |
| IV. | DELUMP | 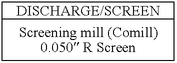 | |
| V. | BLEND | 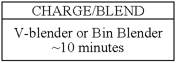 | |
| VI. | LUBRICATE | 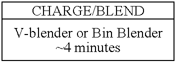 | Add: 6. Magnesium Stearate (non-bovine) |
| VII. | COMPACT | 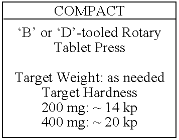 | 100 mg strength = 262.5 mg weight (‘B’) 200 mg strength = 525.0 mg weight (‘B’) 400 mg strength = 1050.0 mg weight (‘D’) |
| VIII. | FILM-COAT | 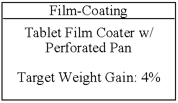 | Add: 7. Purified Water * 8. Opadry II Yellow (22% solids) |

### Example 5

### Manufacture of Tablets Using a High-Shear Wet Granulation Technique

The active ingredient along with portions (or all) of the following ingredients: microcrystalline cellulose, copovidone and crospovidone are mixed together with high-shear force in a pharmaceutical granulation bowl or mixer until a uniform blend results. Granulation fluid (water, with or without dissolved copovidone) is then gradually added while mixing with both the impellor and chopper at medium speed until granules form. Mixing is continued as needed to further densify the granules until a satisfactory endpoint is reached. The resulting wet granulation mass is then processed in a fluid bed dryer or tray-drying oven at 30-60° C. until the moisture level is reduced to a satisfactory endpoint. The dried granules are then passed through a screening mill or hammer mill to produce smaller granules of a more uniform particle size. The resulting dried, sized granulation is then blended together with remaining portions (or all) of the following ingredients: microcrystalline cellulose, copovidone, crospovidone, colloidal silicon dioxide and magnesium stearate. The resulting blend is then compacted on a rotary tablet press to produce tablets which can then be film coated.

### Example 6

Description of Tablet Formulations Containing Formula I Tartrate
Prepared Using Any of the Four Formulation Techniques Including
High-shear Wet Granulation, Fluid Bed Granulation, Dry Powder
Direct Compression and Dry Granulation Direct Compression

| Ingredient | Function | % w/w range | Actual % w/w |
|---|---|---|---|
| PHX1149T | Active drug | 40-90 | 73.1 |
| tartrate | substance | | |
| Microcrystalline | Diluent | 5-35 | 15.2 |
| Cellulose | | | |
| Copovidone | Binder | 0.1-10 | 4.8 |
| Crospovidone | Disintegrant | 0.1-10 | 5.7 |
| Colloidal Silicon | Glidant | 0.05-3 | 0.2 |
| Dioxide | | | |
| Magnesium | Lubricant | 0.1-3 | 1.0 |
| Stearate | | | |
| CORE TABLET WEIGHT TOTAL (Theoretical) | | | 100.0 |
| Opadry ® II | Film coating | 0.1-10% weight | 4% weight |
| 85F92275 | | gain | gain |
| Yellow | | | |

| | |
|---|---|
| COATED TABLET WEIGHT TOTAL (Theoretical) | 104.0 |

Tablet description: Pale yellow modified oval-shaped tablet. Other possible embodiments of shape for a tablet of this size include: oval-shaped, capsule shaped, modified capsule shaped and almond shaped.
Tablets of any strength from 50 mg to 600 mg, such as tablets preferably containing 50 mg, 100 mg, 200 mg or 400 mg of API may be produced by compacting this blend to different target weights.

1. A tablet dosage form for an active pharmaceutical ingredient comprising a compound of formula (I) 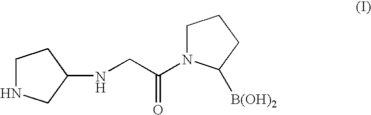
as a tartarate salt, including a solvate, hydrate, tautomer, stereoisomer, or prodrug thereof, comprising:
a diluent comprising microcrystalline cellulose;
a disintegrant comprising crospovidone;
a binder comprising copolyvidone;
a lubricant comprising magnesium stearate; and
a glidant comprising colloidal silicon dioxide;
wherein the dosage form is free of calcium phosphate.

2. The dosage form of claim 1 wherein the active pharmaceutical ingredient comprising a tartarate salt of the compound of formula (I) is substantially anhydrous.

3. The dosage form of claim 1 comprising about 50 mg to about 500 mg of the tartarate salt of the compound of formula (I) on a free base basis.

4. The dosage form of claim 1 comprising about 50 mg, 100 mg, 200 mg, or 400 mg of the tartarate salt of the compound of formula (I) on a free base basis.

5. The dosage form of claim 1 wherein the compound of formula (I) is a compound having the stereochemical configuration (2R)-1-{2-[(3R)-pyrrolidin-3-yl]-acetyl}-pyrrolidine-2-boronic acid.

6. The dosage form of claim 1 wherein the active pharmaceutical ingredient comprises a compound of formula (II) 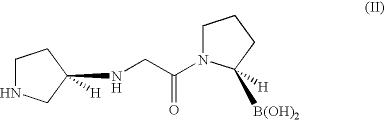
as a tartarate salt, including any solvates, hydrates, tautomers or stereoisomers thereof.

7. The dosage form of claim 6 comprising about 50 mg to about 500 mg of the compound of formula (II) on a free base basis.

8. The dosage form of claim 6 comprising about 50 mg, 100 mg, 200 mg, or 400 mg of the compound of formula (II) on a free base basis.

9. The dosage form of claim 1 wherein the tartarate salt is a monotartarate salt.

10. The dosage form of claim 1 wherein the tartarate salt is a L-tartarate salt.

11. The dosage form of claim 1 wherein the dosage form comprises about 0.1-10 wt % crospovidone, about % 0.05-3% colloidal silicon dioxide, and about 0.1-3% magnesium stearate.

12. The dosage form of claim 1 wherein the tablet is capsule-shaped.

13. The dosage form of claim 1 wherein the tablet is coated with a moisture-repelling coating material.

14. The dosage form of claim 13 wherein the coating material comprises a polymer, preferably polyvinyl alcohol or polyvinyl acetate.

15. (canceled)

16. The dosage form of claim 1 wherein at a temp of 25° C. and a RH of 60%, less than 0.5% impurities derived from the compound of formula (I) are formed over a period of 3 months as determined by HPLC.

17. The dosage form of claim 1 wherein at a temp of 25° C. and a RH of 60%, less than 1% impurities derived from the compound of formula (I) are formed over a period of 6 months as determined by HPLC.

18. The dosage form of claim 1 wherein at a temp of 40° C. and a RH of 75%, less than 0.5% impurities derived from the compound of formula (I) are formed over a period of 3 months as determined by HPLC.

19. The dosage form of claim 1 wherein at a temp of 40° C. and a RH of 75%, less than 1% impurities derived from the compound of formula (I) are formed over a period of 6 months as determined by HPLC.

20. The dosage form of claim 1 formed by a process including a step of fluidized bed granulation or a step of high shear granulation, or both.

21. A method of preparing the dosage form of claim 1, comprising:
milling the compound of formula (I) tartarate salt to provide a milled compound; then,
blending the milled compound with the diluent to provide a blended milled compound; then
in a fluidized bed granulator, granulating the blended milled compound with a solution of the binder in water to provide granules; then
drying the granules to provide dried granules; then
milling and screening the dried granules to provide dried, milled granules; then
blending the dried, milled granules with the glidant, and the lubricant to provide a lubricated blend; then
compressing the lubricated blend in a tablet press to provide the dosage form;
wherein the dosage form is free of calcium phosphate.

22. The method of claim 21 further comprising, after milling the compound of formula (I) tartarate salt, passing the milled compound through a 0033 screen.

23. The method of claim 21 wherein the solution of the binder in water is about a 25% solution of copovidone in water.

24. The method of claim 21 wherein the granules are dried to about 2% to about 8% moisture content.

25. The method of claim 21 wherein the step of granulation comprises top spray granulation.

26. The method of claim 21 wherein the step of blending comprises blending together the dried milled granules, the disintegrant, and the glidant, and then, adding the lubricant and blending in the lubricant.

27. The method of claim 21 wherein the compound of formula (I) tartarate salt is a monotartarate salt.

28. (canceled)

29. The method of claim 21 wherein the tablet press is a rotary tablet press fitted with modified capsule shaped tooling at a target tablet weight.

30. The method of claim 21 further comprising coating the tablets with a moisture-repelling coating material, preferably a material comprising a polymer.

31. A method of preparing the dosage form of claim 1, comprising:
in a high shear granulator, dry mixing the compound of formula (I) tartarate, the diluent, and the binder to provide a dry mix; then
adding water to the dry mix to provide granules; then
drying and milling the granules; then
adding the glidant and the lubricant; then
mixing to provide a lubricated blend; then
compressing the lubricated blend in a tablet press to provide the dosage form;
wherein the dosage form is free of calcium phosphate.

32. The method of claim 31 further comprising adding an additional quantity of the compound of formula (I) tartarate during the step of adding the glidant, and the lubricant.

33. The method of claim 32 wherein the additional quantity of the compound of formula (I) tartarate is about 10% of the weight of an amount of the compound of formula (I) tartarate dry mixed with the diluent and the binder.

34. The method of claim 31 wherein the compound of formula (I) tartarate is a monotartarate.

35. (canceled)

36. The method of claim 31 wherein the tablet press is a rotary tablet press fitted with modified capsule shaped tooling at a target tablet weight.

37. The method of claim 31 further comprising coating the tablets with a moisture-repelling coating material, preferably a material comprising a polymer.

38. A dosage form prepared by the method of claim 21.

39. A dosage form prepared by the method of claim 31.

40. A method of treating a malcondition in a patient wherein inhibition of DPP-IV is indicated, comprising administering the dosage form of claim 1 at a frequency and over a period of time sufficient to provide a beneficial effect to the patient.

41. A method of treating a malcondition in a patient wherein inhibition of DPP-IV is indicated, comprising administering the dosage form prepared by the method of claim 21 at a frequency and over a period of time sufficient to provide a beneficial effect to the patient.

42. A method of treating a malcondition in a patient wherein inhibition of DPP-IV is indicated, comprising administering the dosage form prepared by the method of claim 31 at a frequency and over a period of time sufficient to provide a beneficial effect to the patient.

43. A dosage form of claim 1 further comprising a moisture-protective coating.

44. A method of preparing the dosage form of claim 1, comprising:
dry granulating a combination of the compound of formula (I) tartarate salt and the diluent to form granules,
grinding the granules to form a powder,
combining the powder with the disintegrant, binder, glidant and lubricant to form a blend,
compressing the blend in a tablet press to provide the dosage form;
wherein the dosage form is free of calcium phosphate.

45. A method of preparing the dosage form of claim 1 or 6, comprising
first, separately, passing each of: the compound of formula (I) or (II) respectively as a tartrate salt, the diluent, the disintegrant, the binder, the lubricant, and the glidant, through a screening mill;
then, mixing the screened compound of formula (II), the screened diluent, and the screened binder in a blender to provide a homogeneous powder;
then, adding the screened disintegrant and the screened glidant to the homogeneous powder, and then performing additional mixing in the blender;
then, mixing with the lubricant in a blender;
then compressing in a tablet press to provide an uncoated dosage form;
then spraying a coating onto the uncoated dosage form to provide a coated dosage form;
wherein the dosage form is free of calcium phosphate.

46. A dosage form prepared by the method of any of claim 21, 31, 44, or 45, comprising about 50 mg to about 500 mg on a free base basis of the compound of formula (I) or (II), respectively, as a tartrate salt.
